(19) United States
(12) Patent Application Publication
(10) Pub. No.: US 2022/0349866 A1
(43) Pub. Date: Nov. 3, 2022

(54) GAS SENSING DEVICE DESIGNED FOR ENVIRONMENTAL DECONTAMINATION WITH SIMULTANEOUS ALERT EMISSION

(71) Applicants: José Marcos NABHAN, Cianorte - PR (BR); Vinicius Batistela NICOLETTI, Cianorte - PR (BR)

(72) Inventors: José Marcos NABHAN, Cianorte - PR (BR); Vinicius Batistela NICOLETTI, Cianorte - PR (BR)

(21) Appl. No.: 17/609,768

(22) PCT Filed: Jan. 24, 2020
(86) PCT No.: PCT/BR2020/050015

§ 371 Date: Nov. 8, 2021

(30) Foreign Application Priority Data
Jan. 22, 2020 (BR) BR 20 2020 001336

(51) Int. Cl. G01N 33/00; G08B 21/14

(52) U.S. Cl. CPC G01N 33/004; G01N 33/0063; G08B 21/14

(57) ABSTRACT

A gas sensing device designed for environmental decontamination with simultaneous alert emission comprising a safety system for occupants of closed environments, including vehicles and aircraft, that simultaneously activates the ventilation/exhaust of the enclosure and emits visual and audible signals with alerts and notifications over a mobile network, and a mechanism for opening windows and unlocking doors once the presence of human beings or animals and concentrations primarily of CO2 posing a health risk are detected.

### FIELD OF THE INVENTION

Oxide present in the atmosphere, including by-product of the entire respiratory process, carbon dioxide (CO₂), can be extremely harmful to health in large concentrations.

In fact, the records of children drying after being left inside vehicles under the sun are alarming, even after only short periods of time.

In the breathing process, the body absorbs oxygen and releases CO₂. In closed environments, such as motor vehicles, for example, there is a risk of rebreathing gas and, therefore, of increased levels of concentration of the substance in the blood, leading to symptoms such as headache, drowsiness, eye irritation, change in humor, in addition to contamination through viral diseases when good ventilation in the environment is not available.

That said, this disclosure is inherent to a device that monitors the rate of carbon dioxide (CO₂) inside the cabin of motor vehicles, decontaminating the environment through ventilation mechanisms, opening windows and unlocking the doors of the vehicle itself and sending alerts through audible signals from the device itself, and light and audible signals from the vehicle's own optical and audible systems as well as calls and messages to phones previously registered in the embedded app.

Breathing naturally produces CO₂, which means that the presence of people or animals inside the cabin is detectable by the sensor even before decontamination and an alert is sent.

With the vehicle running or simply turned on and in the presence of at least one occupant, in approximately 5 minutes the gas concentration rate will exceed 1000 ppm, this is the tolerance limit recommended by the WHO.

When the vehicle's ignition key is turned on, the device detects a concentration above 1000 ppm of CO₂ in the environment, alerting the user/occupant to the activation of the exhaust or ventilation system through a continuous and then intermittent beep, avoiding the risk of rebreathing and symptoms such as drowsiness.

When the vehicle is turned off, the device remains in operation in order to detect the presence of living beings inside the closed cabin, avoiding the occurrence of accidents involving especially children, the elderly and animals that may be left inside the vehicle, activating, when applicable, the mechanisms for opening windows and unlocking the doors after sending messages to smart devices, such as previously registered smartphones, for example.

### DESCRIPTION OF THE STATE OF THE ART

High temperatures with increasing and more frequent occurrence, including in countries of the northern hemisphere, associated with numerous records of deaths of children, the elderly and animals inside vehicles parked under the sun, have increased the demand for effective technologies to protect closed environments from airborne contamination by carbon dioxide (CO₂).

Instruments and means that somehow relate to this specific protection are not rare in common practice, contemplating the most diverse solutions.

In searches carried out in the state of the art, it was possible to find documents involving some of these concepts, of which, those whose operation obey dissimilar concepts and not similar to the ones in this report, the following have been selected:

Document US1999027788 discloses a sensor for detecting gases, comprising a housing and an active element inside the housing. The active element is surrounded by a porous insulating material with a bulk density not exceeding 0.15 g/cm\hat{ }3\hat{ }. Another gas sensor comprises an active element surrounded by a porous insulating material with a surface area of not more than approximately 200 m\hat{ }2\hat{ }/cm\hat{ }3\hat{ }. Another gas sensor comprises a copper compound positioned so that the gas contacts the copper compound before contacting the active element. Another gas sensor comprises an active element surrounded by a porous material with an average pore size of at least approximately 100<134>.

Document DK2005000381 discloses IV sensor (1), especially a CO₂ sensor, with a filter arrangement (6), behind which is a detector (7) and an assessment device (8) which is connected with the detector arrangement (7), the filter arrangement (6) having a first filter (9) and a second filter (10), which are configured as passband filters and respectively have one pass and of which the first filter (9) passes a predetermined IV band and the second filter (10) does not, and the detector arrangement has two detectors (14, 15), of which each is combined with a filter (9, 10). It is intended to simplify the application of such an IV sensor. For this purpose, it is foreseen that the passband of one filter (10) is arranged within the passband of the other filter (9) and the assessment device (8) takes the difference between the signals (S1, S2) of the detectors (14, 15) and adapts it to the signal (S1) of a detector (14)”. (free translation)

Document BR PI0601721-5 A2 teaches a process for determining ozone in the atmosphere, through spectrophotometric and spectrofluorometric measurements, using filters impregnated with indigo blue to collect the gas. Spectrophotometric analyses performed at 600 nm, the process most commonly described in the literature, showed to be significantly sensitive to the action of interferents, compared to measurements made at 250 nm. These data allow us to conclude that more reliable ozone determinations are obtained in the ultraviolet region. Using a process whose determination is spectrofluorometric, the measurements were quite sensitive, since it is possible to distinguish between close concentrations, which are often not differentiated by the spectrophotometric process. In addition, the selectivity was also superior to the spectrophotometric technique, since there was no interference from any other gaseous species, even when present in a concentration much higher than that of ozone.

Document DE2008000422 teaches a generic method for detecting and identifying gases in interior spaces of the aircraft and an associated device, small and manageable, has a simple design and allows immediate and simultaneous detection and identification of the gases to be examined. This is achieved in the fact that the air supply from the interior space of the aircraft (20) is fed to a measuring device (1) and the measurement results of the measuring device (1) are analyzed by means of mathematical methods. Such methods and associated devices to detect and identify gases in the interior spaces of aircraft are used to detect and verify gases, particularly odors and explosive gases and/or gases harmful to human health.

Document D32010051043 deals with a sensor chip (1030) for gas, equipped with cells (200) to emit and receive ultrasound and is set to a sufficiently large frequency range and to measure the concentration of at least one of the gas components based on at least two responses within the range. The frequency range can be achieved by varying the size of the cell membranes (230), varying the bias voltages and/or varying the air pressure. The sensor chip can be applied, for example, in capnography. A measurement air chamber (515) is implemented in the airway (400) and it and/or the airway can be designed to reduce turbulence in exhaled breath (120) subject to ultrasound interrogation. The chip (1030) can be implemented as independent in monitoring parameters, avoiding the need for off-chip sensors.

Document EP2011068984 reveals an air treatment device comprising of an air purification unit, configured to purify the air; an air sensor configured to measure a first amount of air and provide a measurement output, wherein the first amount of air comprises air purified by the air purification unit; and a processor configured to generate a first value based on the measurement output of the air sensor so as to calibrate the air sensor. With the air treatment device of an embodiment of the invention, clean air, i.e., zero air, is generated locally by the air treatment device in order to calibrate the air sensor, without the need to externally generate the zero air, which provides convenience for the user or other operators performing the calibration of the air handling device's air sensor. Another embodiment of the invention also provides a method for calibrating an air sensor of an air treatment device. The method comprises the steps of: purifying the air using the air treatment device; and measuring a first amount of air using the air sensor to obtain a first value so as to calibrate the air sensor, wherein the first amount of air comprises purified air.

Document D32012053501 deals with the method to selectively detect the concentration of a target gas in polluted ambient air, comprising: provision of a target gas sensor (220) sensitive to the target gas; providing a first flow of gas derived from ambient air, from which the first flow of the target gas is substantially removed; providing a second gas stream derived from ambient air comprising substantially the same concentration of target gas as ambient air; exposing the target gas sensor to the first gas flow for a first time interval and obtaining a first output signal (Smf) from the sensor; exposing the target gas sensor to the second gas flow for a second time interval not overlapping the first time interval and obtaining a second output signal (Smu); calculate the difference (S?) between the first and second output signals; calculate the target gas concentration from the calculated signal difference.

Document US2016/0103111 teaches a vehicle occupant safety system includes a carbon dioxide (CO₂) sensor and a controller. The CO₂ sensor is configured to determine a concentration of CO₂ in a vehicle's passenger cabin while the vehicle is not in operation, i.e., parked. The controller is configured to determine which passenger cabin is occupied by an occupant based on the concentration of CO₂ in the passenger cabin while the vehicle is not in operation. If CO₂ concentration is a concern while the vehicle is not in operation, the system can respond by activating a means of notification, such as a horn or vehicle alarm, or by ventilating the passenger cabin, opening windows or activating a fan of the vehicle's HVAC system.

Knowing the documents inherent to the state of the art, it is inferred that all have the primary purpose of detecting and identifying gases, including CO₂, sometimes in the atmosphere, sometimes in certain environments, as is the case of documents DE2008000422 and US2016/0103111A1, respectively applied in the interior spaces of airplanes and in the cabin of motor vehicles when parked.

Other documents demonstrate similar functionalities regardless of the environment to which they apply, as in the case of patents US1999027788, DK2005000381, D32012053501 and D32010051043, this apparently for the airway, or in the atmosphere, as seen in document BR PI0601721-5A2, or, also, in aircraft interior environments, in document DE200800042.

When the focus is on automotive vehicles, the operation is determined by the fact that the vehicle's engine is turned off, as seen from document US2016/0103111A1.

The document EP2011068984 refers to air purification process and does not specify the environment of use, i.e., if in a closed environment or in the atmosphere, while the document EP2014061811 refers to the transcutaneous measurement of a gas concentration.

Although the documents found present a similar general concept, proposing a similar end-objective, the technical state is improved in the object now reported, which proposes the decontamination of motor vehicle cabins by monitoring the concentration rate of the carbon dioxide (CO₂) (2) and detecting the presence of living beings inside the cabin, emitting alarm signals (11) and activating the fan/exhauster, opening windows (5) and unlocking the doors (8).

When the ignition key is turned off, the sensor (1) will monitor the environment by carrying out, if the gas concentration rate is above the pre-programmed limit with an addition of 400 ppm, the emission sound signal functions (11) to alert and, if applicable, activate the fan/exhaust (6) and send calls/messages to registered telephones, activating the optical (7) and audible set of the vehicle itself. If there is no human intervention 5 minutes after the alert system is initiated, the device (1) will activate the mechanisms to open windows (5) and unlock the doors (8).

As a result, although they presuppose the existence of priorities such as those found, the proposed improvements provide significant functional improvements, as, when the vehicle is in motion, they trigger a sound signal for 20 seconds so that the ventilation system is triggered manually by the user (6), or, in case of inaction of the user/occupant, it automatically activates said ventilation system (6), to prevent rebreathing the concentrated gas in the vehicle cabin. When the vehicle is turned off and upon detection of the presence of living beings in the closed cabin, the device (1) monitors the environment by measuring any increase in the concentration of CO₂ (2) up to the pre-programmed limit with an addition of 400 ppm exactly for the sensor (1) detects the concentration before the limit recommended by the WHO of 1000 ppm and performs, if necessary, the functions of triggering the sound signal (11) for 20 seconds and visual for another 20 seconds until the occupant/user activates the vehicle ignition key, adding 400 ppm again and so on, and activating the vehicle's fan (6) for air renewal which will follow, when necessary, by making phone calls and sending alert messages and activating the optical and sound set of the vehicle itself and, after the pre-programmed time of 5 minutes, without human intervention, the mechanisms for opening windows (5) and unlocking the doors (8), which is not revealed in the state of the art.

### SUMMARY OF THE OBJECT OF THE PATENT

A safety device (1 is provided to monitor the CO₂ rate (2) in motor vehicle cabins, which includes: a carbon dioxide (CO₂) sensor (1) that detects the presence of people or animals and measures the gas concentration; an actuating command to activate the ventilation/exhaustion (6) of the vehicle; display (11) indicative of gas concentration measurement and visual and audible alarm; an actuating command for opening windows (5) and unlocking the doors (8); and an alarm system that can be communicated with the embedded computer and ignition keys, as for most modern vehicles, and with previously registered smart devices, such as smartphones, for example, with wide area network capability or Bluetooth.

The sensor (1) is configured to first detect the presence of humans or animals in the environment. If CO₂ (2) is detected in a harmful concentration, visual and audible electronic warnings are issued from the device itself, especially from the display (11) indicative of the measurement of the gas concentration, and the set can be use the optical and audible (7) system of the vehicle itself or customized alarm, for example, and effectively using media devices, either from the vehicle, or previously registered smart devices, such as smartphones, for example, including the keys of more modern cars, simultaneously with the ventilation/exhaustion operations (6) and opening windows (5) and unlocking doors (8) also configured.

Other features and functional improvements will be defined more clearly from the Detailed Description of the preferred embodiment and with reference to the following drawing examples.

### DESCRIPTION OF THE DRAWINGS

The characterization of the improvements under discussion is given through a schematic drawing of the proposed solution, which expresses the preferred ways of achieving the object, supporting this description through numerical and consecutive references.

Therefore:

FIG. 1 in the block diagram describes the components and operations from the sensor (1). In the right column, the sequence of operations starting with the detection (2) of the presence of occupants in the environment and the concentration of CO₂ at levels equal to or greater than the determined value, when the sensor (1) indicates on the display (11) the value of the gas concentration (ppm). If the concentration is greater than 1000 ppm, the device will emit a continuous beep on the display (11), becoming intermittent and then illuminate as the concentration increases until the user/occupant activates the fan/exhauster (6), when the vehicle is in operation.

If the vehicle is not in operation, the sensor (1) will monitor the environment by measuring any increase in the concentration of CO₂ up to the pre-programmed limit, from when, upon detection of the presence of animals or humans and if the occupant/user does not act, it activates the fan/exhaust (6) and, if necessary, the other operations as explained below. The same figure also informs the presence of a battery (4).

### DETAILED DESCRIPTION OF THE INVENTION

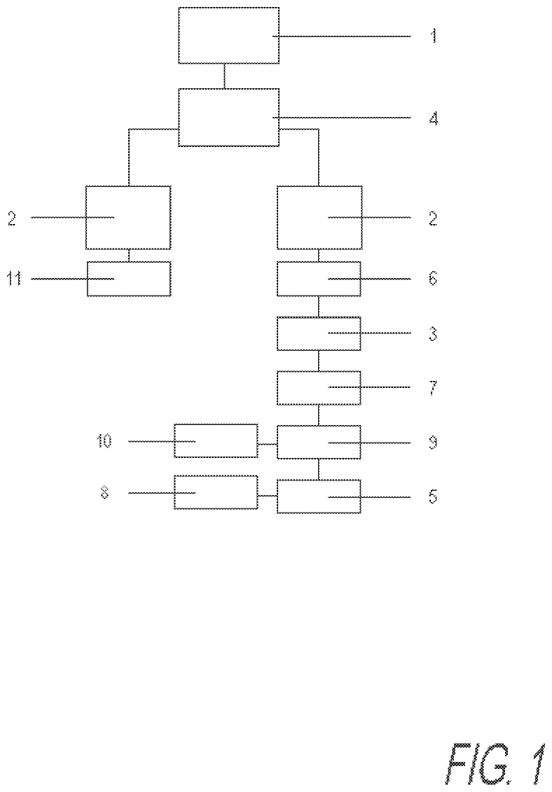

FIG. 1 illustrates a non-limiting example of a safety device (1) for cabins of motor vehicles when occupied by humans or animals. The device includes a sensor (1) for carbon dioxide (CO₂) (2) configured to determine a concentration of CO₂ (2) at pre-set levels. Appropriate CO₂ sensors are available in the state of the art, and the device in question takes advantage of the reliability of this type of detection to provide additional safety in the use of motor vehicles, which can be installed even in more modern vehicles, equipped with embedded computers and in that the ignition is given by an electronic or physical device (9), with which the sensor (1) communicates, whether the vehicle is in operation or not.

Said device (1) measures the concentration of CO₂ (2) and controls the decontamination of the air. As CO₂ is a by-product of respiration, harmful concentrations can occur in a closed environment. The literature suggests that concentrations of more than one part per million (1000 ppm) can lead to drowsiness. Without sufficient air renewal, the concentration of gas will increase and will certainly reach intolerable levels, which have the potential to cause harm to health and even accidents.

Considering the molecular density of CO₂ and the use of the device (1) in automotive vehicles, it is appropriate for the sensor (1) to be installed in the lower regions of the cabin. The device should preferably be installed in the center panel of the vehicle.

Once the presence of occupants in the environment and the concentration of CO₂ (2) at levels equal to or greater than the determined value is detected, the sensor (1) will indicate (11) the gas concentration value (ppm) on the display. If the concentration is greater than 1000 ppm, the device will emit a continuous beep on the display (11) which will become intermittent and then illuminate as the gas concentration increases and until the user/occupant activates ventilation/exhaustion (6) or open windows (5) if the vehicle is in operation.

If the vehicle is not in operation, the sensor (1) will monitor the environment by measuring any increase in the concentration of CO₂ up to the pre-programmed limit with an addition of 400 ppm so that, if the environment contains, for example, 800 ppm when the vehicle is turned off, the sensor (1) reads 1200 ppm, performing the subsequent functions of: a) triggering an audible signal (11) alert for 20 seconds and visual for another 20 seconds, until the vehicle occupant activates the ignition key, adding 400 ppm again and so on; b) activating the vehicle's fan/exhaust (6) to renewal the air. Once the increase in the concentration of gas in the cabin is identified, the device will emit an intermittent sound signal for 20 seconds. If the air is not renewed through manual action, the device (1) will activate the vehicle's mechanical ventilation (6) automatically and, subsequently, calls to registered cell phones and send messages with the geolocation to other registered contacts, activating the optical (7) and audible set of the vehicle itself.

If there is not human intervention to re-establish the CO₂ concentration levels (2) 5 minutes after the alert system is activated, the device will trigger the mechanism for opening windows and unlocking the doors (5 and 8). Also, the device (1), unless the function is disabled, will call emergency services via GPS.

The device (1) will record the CO₂ concentration level (2) in the cabin every 48 hours, generating a hazard verification report.

Although the improvements in discussion have been described in terms of their preferred embodiments, they are not intended to be limited, but only to the extent set forth in the claims below.

The literature reports that concentrations greater than one part per million (1000 ppm) have harmful potential. Without fresh air, occupants in the vehicle's cabin, for example, especially children, the elderly and animals, would be at a very serious risk.

In contrast, in environments without the presence of people or animals, the variation in CO₂ levels is very low. Some time without occupants is enough for gas concentration levels to gradually drop to normal levels.

Thus, FIG. 1 illustrates a non-limiting example of a safety device (1) for occupants of motor vehicle cabins that includes a CO₂ sensor (1) configured to determine the presence of people or animals.

The sensor itself (1) is configured to determine if the environment is occupied by people or animals. As used herein, the terms “occupant” and “user” designate people and animals, such as, but not limited to, humans, dogs and others that may occupy the cabins of motor vehicles.

The device (1) takes advantage of and integrates devices based on existing microcomputers in the vehicle (3, 6, 7, 9 and 11).

Thus, the sensor device (1) measures the concentration of CO₂ (2) and provides an indication of the air quality in the environment via display (11) or embedded computer.

The example in FIG. 1 proposes that the device (1) is used in a closed environment, which can be the cabin of any vehicle, and may include a processor not shown, such as a microprocessor or other control circuit. Such circuits can be analog or digital, including application-specific in the cabin

The sensor device (1) may include non-volatile memory, such as electronically erasable reprogrammable read memory (EEPROM) to store one or more routines, limits and captured data, which can be performed by the processor in view of the steps to determine if there are any occupants in the cabin.

It is known that CO2 has a higher molecular density than the main components of the typical atmosphere, which are N2 and 02 and means it is advantageous for the sensor (1) to be located in the lower regions of the cabin of the motor vehicle. Preferably, the device should be installed on the dashboard.

In addition to activating the means of notification (3, 7, 10, 11), the sensor device (1) includes an actuating command to activate the ventilation/exhaustion system (6) operated by a controller in common use and not shown. This ventilation/exhaust system (6) includes window (5) and door (8) equipped with actuators capable of opening them.

1.—Gas sensor device for discounting environmental action and simultaneous emission of alert
defined to determine a concentration of CO2, comprising
a sensor of carbon dioxide (CO2),
a fan of the air in a certain closed environment,
an optical assembly, and
smart devices registered for the emission of electronic and call signals served by emergency via GPS,
obtained by the sensor simultaneously activating the exhaust system ventilated, the device specific audible alarm and the mechanism for opening windows and unlocking doors.
